(19) United States
(12) Patent Application Publication
(10) Pub. No.: US 2024/0402194 A1
(43) Pub. Date: Dec. 5, 2024

(54) IMMUNOASSAY FOR DETECTING NEURODEGENERATIVE DISEASES PATHOLOGY IN BIOLOGICAL FLUIDS

(71) Applicant: Qatar Foundation for Education, Science and Community Development, Doha (QA)

(72) Inventor: Omar El Agnaf, Doha (QA)

(21) Appl. No.: 18/734,505

(22) Filed: Jun. 5, 2024

Related U.S. Application Data
(60) Provisional application No. 63/471,128, filed on Jun. 5, 2023.

(51) Int. Cl. G01N 33/68; C07K 16/18; G01N 33/536

(52) U.S. Cl. CPC G01N 33/6896; C07K 16/18; G01N 33/536; G01N 2800/2821; G01N 2800/2835

(57) ABSTRACT

Antibodies and diagnostic assays for detecting synucleinopathies are provided. The assay is specific and compatible with different biological samples (brain, cerebrospinal fluid, blood, saliva, urine, skin, nasal swabs, or feces).

### CROSS REFERENCE TO RELATED APPLICATIONS

This application claims the benefit of U.S. Provisional Application No. 63/471,128 filed on Jun. 5, 2023, which is incorporated herein by reference in its entirety.

### SEQUENCE LISTING

This application contains a sequence listing having the filename 5600234-01173US_Sequence_Listing.xml, which is 9 KB in size, and was created on Jun. 4, 2024. The entire content of this sequence listing is incorporated herein by reference.

### FIELD

The present disclosure relates to methods for detecting neurodegenerative diseases.

### BACKGROUND

The pathogenesis of Parkinson's disease (PD), dementia with Lewy bodies (DLB), and multiple system atrophy (MSA) is closely associated with the misfolding and aggregation of the protein α-synuclein into toxic insoluble fibrils in the affected regions of the brain. Collectively known as synucleinopathies, these diseases pose significant diagnostic challenges due to the lack of disease-specific clinical criteria. Despite advances in understanding these conditions, the identification of reliable and disease-specific biomarkers remains an unmet need.

### SUMMARY

Described herein is a diagnostic assay that utilizes conformation-specific monoclonal antibodies that target α-synuclein aggregates and provides analysis of disease-related α-synuclein pathology. The diagnostic assays allow for the detection and assessment of α-synuclein pathology in various biological samples, including, but not limited to, brain, cerebrospinal fluid, blood plasma or serum, saliva, urine, skin, nasal swabs, and feces.

Disclosed herein are antibodies, or fragments thereof, specific for α-synuclein aggregates, wherein the antibody has a variable light (VL) and a variable heavy (VH) amino acid sequence comprising: (a) a VL sequence of SEQ ID NO:2 and a VH sequence of SEQ ID NO:3; or (b) a VL sequence of SEQ ID NO:4 and a VH sequence of SEQ ID NO:5. In some embodiments, the antibody comprises a VL sequence of SEQ ID NO:2 and a VH sequence of SEQ ID NO:3.

Disclosed herein are methods for detecting α-synuclein aggregates in a sample from a subject, the method comprising: (a) contacting the sample with a first antibody specific for α-synuclein aggregates to form first antibody-α-synuclein complexes, wherein the first antibody is an antibody disclosed herein; and (b) detecting the α-synuclein-first antibody complex; wherein the presence of α-synuclein aggregates in the sample is indicative of a synucleinopathies in the subject. In some embodiments, the synucleinopathies is selected from Parkinson's disease, dementia with Lewy bodies, multiple system atrophy, and Alzheimer's disease. In some embodiments, the first antibody comprises a VL sequence of SEQ ID NO:2 and a VH sequence of SEQ ID NO:3. In some embodiments, the first antibody comprises a VL sequence of SEQ ID NO: 4 and a VH sequence of SEQ ID NO:5. In some embodiments, the sample is selected from brain, cerebrospinal fluid, blood plasma or serum, saliva, urine, skin, nasal swab, or feces. In some embodiments, the synucleinopathies is prodromal Parkinson's disease, dementia with Lewy bodies, or multiple system atrophy. In some embodiments, α-synuclein-first antibody complexes are detected with a second antibody specific for α-synuclein aggregates. The first antibody and the second antibody can be different antibodies or the same antibody.

Also disclosed herein are assays for detecting a neurodegenerative disease characterized by protein misfolding in a subject, the assay comprising: (a) obtaining a sample from the subject; (b) contacting the sample with a first antibody specific for α-synuclein to form first antibody-α-synuclein complexes, wherein the first antibody is an antibody disclosed herein; and (c) detecting the α-synuclein-first antibody complex; wherein the presence of α-synuclein aggregates in the sample is indicative of a neurodegenerative disease characterized by protein misfolding in the subject. In some embodiments, the neurodegenerative diseases characterized by protein misfolding is selected from tauopathies, Alzheimer's disease, frontotemporal dementia, Pick's disease, progressive supranuclear palsy, corticobasal degeneration, Huntington's disease, amyotrophic lateral sclerosis, motor neuron diseases, spinocerebellar ataxia, psychosis, schizophrenia, Creutzfeldt-Jacob disease, and spinal muscular atrophy. In some embodiments, the sample is selected from brain, cerebrospinal fluid, blood plasma or serum, saliva, urine, skin, nasal swab, and feces. In some embodiments, the first antibody comprises a VL sequence of SEQ ID NO:2 and a VH sequence of SEQ ID NO:3. In some embodiments, α-synuclein-first antibody complexes are detected with a second antibody specific for α-synuclein aggregates. The first antibody and the second antibody can be different antibodies or the same antibody.

Also disclosed herein is an immunoassay kit comprising at least one α-synuclein aggregate-specific antibody disclosed herein and instructions for performing an assay using the antibody.

Also disclosed herein is an antibody, or a fragment thereof, disclosed herein, for use as a medicament.

Also disclosed herein are pharmaceutical compositions comprising an antibody, or fragment thereof, disclosed herein and a pharmaceutically acceptable diluent or carrier.

Also disclosed herein are methods for treating a neurodegenerative disorder with α-synuclein pathology in a subject in need thereof comprising administering an antibody, or fragment thereof, disclosed herein to the subject. In some embodiments, the neurodegenerative disorder with α-synuclein pathology is selected from Parkinson's disease, Alzheimer's disease, dementia with Lewy bodies, and multiple system atrophy. In some embodiments, the antibody comprises a VL sequence of SEQ ID NO:2 and a VH sequence of SEQ ID NO:3.

Also disclosed herein a e methods for detecting α-synuclein in a sample from a subject, the method comprising: a) coating wells of a plate with a first antibody targeting α-synuclein aggregates; b) adding a mixture of a biological sample and recombinant full-length or fragment of α-synuclein monomers to the coated plate to form first antibody-α-synuclein aggregate complexes; c) incubating the plate under shaking conditions or continuous mixing; and d) detecting the first antibody-α-synuclein aggregate complexes; wherein the first antibody targeting α-synuclein aggregates is an antibody disclosed herein. In some embodiments, α-synuclein-first antibody complexes are detected with a second antibody specific for α-synuclein aggregates. The first antibody and the second antibody can be different antibodies or the same antibody.

Also disclosed herein are assays for detecting a neurodegenerative disease characterized by protein misfolding in a subject, the assay comprising: a) coating wells of a plate with a first antibody targeting α-synuclein aggregates; b) adding a mixture of biological sample and recombinant full-length or fragment of α-synuclein monomers to the coated plate to form first antibody-α-synuclein aggregate complexes; c) initiating the seeding and protein amplification process incubating the plate under shaking conditions or continuous mixing; and d) detecting the first antibody-α-synuclein aggregate complexes; wherein the antibody targeting α-synuclein aggregates is an antibody disclosed herein. In some embodiments, α-synuclein-first antibody complexes are detected with a second antibody specific for α-synuclein aggregates. The first antibody and the second antibody can be different antibodies or the same antibody.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1A depicts a diagrammatic representation of the assay. The top panel of the diagram presents a conventional sandwich ELISA protocol that employs a conformation-specific anti-α-synuclein (α-syn) monoclonal antibody (mAb) (2A1) without the seeding amplification step. The bottom panel shows the same assay but with an amplification step.

FIG. 1B depicts a scatter plot that illustrates the seeding effect of α-synuclein seeds observed in a pilot study of BH samples from PD patients and healthy controls.

FIG. 1C depicts a scatter plot that illustrates the seeding effect of α-synuclein seeds observed in a pilot study of CSF samples from PD patients and healthy controls.

FIG. 2A illustrates the sensitivity of the assays described herein by using different dilutions, between 1:200 and 1:2042000, of BH obtained from PD, DLB and MSA subjects.

FIG. 2B depicts testing of α-synuclein pre-formed fibrils (PFFs) at different concentrations in control BH and artificial CSF (ACSF).

FIG. 2C depicts inter-assay variability plots of 4-hydroxy-2-nonenal (HNE) α-synuclein oligomer standards tested over three non-consecutive days.

FIG. 2D depicts inter-assay variability plots of BH and CSF samples tested over two non-consecutive days.

FIG. 3A depicts assay validation in a larger cohort of samples of BH from different synucleinopathies PD, DLB, and MSA. Scatter plot and receiver operating characteristic (ROC) analysis of measured seeded α-synuclein levels in BH samples of PD, DLB, and MSA vs control.

FIG. 3B depicts a scatter plot depicting the measured levels of seeded α-synuclein in CSF samples from a larger cohort consisting of PD patients and healthy controls.

FIG. 4A depicts immunoassay specificity for synucleinopathies with a scatter plot of measured seeded α-synuclein levels in CSF samples from synucleinopathies (PD, MSA, DLB, and PD dementia (PDD)) compared to non-synucleinopathies (progressive supranuclear palsy (PSP), Alzheimer's Disease (AD), vascular dementia (VD), and mild cognitive impairment MCI).

FIG. 4B depicts ROC curve analysis of diagnostic performance of assay in PD, PDD, DLB, and MSA vs PSP.

FIG. 5A depicts scatter plot and ROC curve analyses of seeded α-synuclein in validation cohort of CSF samples from LRRK2 cohort with sPD, manifesting (P8PD) and non-manifesting (P8M+) LRRK2 mutation carriers vs healthy controls.

FIG. 5B depicts scatter plot and ROC curve analyses of seeded α-synuclein in patients with prodromal cases of PD (RBD).

### DETAILED DESCRIPTION

Synucleins are a family of small proteins, about 14 kDa that are expressed at high levels in nervous tissues. The three members of the family are α-, β-, and γ-synucleins. α-Synuclein is expressed mainly in brain tissues and is primarily located at the presynpatic terminal of neurons. The primary structure of the human form of α-synuclein consists of a 140 amino acid polypeptide having the amino acid sequence of SEQ ID NO:1 (UniProtK Accession No. P37840). α-Synuclein normally exists as a soluble monomeric protein but can adopt several different folded confirmations depending on its environment. α-Synuclein aggregates include, but are not limited to amyloid fibrils, protofibrils, or oligomers. Monomeric α-synuclein can also aggregate into oligomers and into higher molecular weight insoluble fibrils.

Diseases associated with abnormalities in synucleins are often referred to as the synucleinopathies. Synucleinopathies include the neurodegenerative conditions, Parkinson's disease (PD), dementia with Lewy bodies (DLB) and multiple system atrophy (MSA). In synucleinopathies it has been shown that soluble α-synuclein oligomers in brain homogenates of PD and DLB are elevated compared to normal brains. In addition, the neuropathologic lesions (Lewy bodies) that often characterize the end stage of PD and DLB have largely been found to be composed of fibrillar α-synuclein deposits.

Disclosed herein are diagnostic assays that addresses a critical gap in diagnostics and therapeutics for synucleinopathies. The assays leverage conformation-specific monoclonal antibodies for α-synuclein aggregates to provide quantitative, disease-specific, and highly sensitive analysis of disease-related α-synuclein oligomers. This approach allows for the detection and assessment of α-synuclein pathology in various biological samples, including brain, cerebrospinal fluid (CSF), blood plasma or serum, saliva, urine, skin, nasal swabs, or feces.

The presently disclosed assays target synucleinopathies including, but not limited to, PD, DLB, and MSA and α-synuclein co-pathologies such as Alzheimer's disease and neurodegenerative diseases characterized by protein misfolding including, but not limited to, tauopathies (comprising accumulation of tau aggregates), including Alzheimer's disease, frontotemporal dementia, Pick's disease, progressive supranuclear palsy, corticobasal degeneration, as well as Huntington's disease, amyotrophic lateral sclerosis, motor neuron diseases, spinocerebellar ataxia, psychosis, schizophrenia, Creutzfeldt-Jacob disease, and spinal muscular atrophy.

Thus, disclosed herein are antibodies having high binding affinity to α-synuclein aggregates and low binding affinity to α-synuclein monomers as well as assays for α-synuclein aggregates. The antibodies have an increased affinity to bind to α-synuclein aggregates compared to monomeric α-synuclein forms. Also within the scope of the present antibodies are antibody fragments having a high binding affinity for α-synuclein aggregates and low binding affinity for α-synuclein monomers. In addition to the antibodies disclosed herein, suitable antibodies are disclosed in U.S. Pat. No. 9,534,044 and U.S. Patent Publication US2023/0365665, both of which are incorporated by reference for all they disclose regarding antibodies to α-synuclein.

Unless otherwise stated the term α-synuclein aggregates is intended to cover early soluble aggregates forms of α-synuclein (such as low and high molecular weight soluble oligomers, including protofibrils) and mature insoluble aggregates forms of α-synuclein (such as mature fibrils). In particular, the antibodies and fragments thereof have high binding affinity to α-synuclein fibrils and high binding affinity to α-synuclein oligomers.

Having a high affinity for α-synuclein aggregates means that the antibodies or fragments exhibit a dissociation constant, Kd of less of than 10⁻⁷M for α-synuclein aggregates. In some embodiments, the antibody exhibits a Kd of less than 10⁻⁸M, or less than 10⁻⁹M, or less than 10⁻¹⁰M, or even less than 10⁻¹¹M. Preferably the α-synuclein is human α-synuclein.

Fibrils are insoluble higher molecular weight aggregated forms of α-synuclein.

Soluble oligomeric forms of α-synuclein come in a variety of sizes and morphologies and includes dimer, trimers, tetramers and multimers. Protofibrils are an intermediate step in the pathway to the formation of the α-synuclein fibrils from the monomeric forms. When the term oligomeric forms of α-synuclein is used this is also intended to include protofibrils.

A fragment thereof of the antibodies means active fragments thereof, i.e. fragments having the same characteristics that are used for the definition of an antibody according to the invention, namely high affinity for α-synuclein aggregates and low binding affinity to α-synuclein monomers. For convenience when the term antibody is used, fragments thereof exhibiting the same characteristic are also being considered.

Having a low binding affinity for α-synuclein monomers means that the binding of an antibody or fragment to α-synuclein monomers is at least 100 times less than the binding to α-synuclein aggregates, about 500 less, or about a 1000 times lower binding affinity to α-synuclein monomers compared to α-synuclein aggregates. In some embodiments, the antibody or fragment thereof has a dissociation constant, Kd, of more than 10⁻⁵M for monomeric α-synuclein. In some embodiments, the antibodies may have a higher affinity for α-synuclein fibrils than for oligomeric forms of α-synuclein.

The binding affinities of the antibodies can be determined by using a variety of methods recognized in the art including, isothermal calorimetry and surface plasmon resonance-based approaches. Binding can also be evaluated using immunoassays such as ELISA or RIAs. Preferably the binding affinity is determined using surface plasmon resonance assays using a BIACore™ X-100.

In some embodiments, the antibodies are conformational antibodies. The antibodies recognize conformational epitopes, i.e. the epitope the antibody recognizes includes the tertiary structure of the aggregates of α-synuclein. In some embodiments, the antibodies bind more strongly to the α-synuclein fibrils than to any of the linear peptide epitopes as described herein. In particular, the binding of the antibodies is at least 100 times higher to the α-synuclein fibrils than to the linear peptide epitopes, 500 times more, or 1000 times more.

The antibodies disclosed herein may bind weakly to an epitope within the amino acid region 125-133 of α-synuclein. By binding weakly means that the binding affinity of the antibodies disclosed herein is at least 100 less to an epitope within the amino acid region 125-133 of α-synuclein than the binding affinity of the antibodies to α-synuclein aggregates, such as at least 100 times less than to α-synuclein fibrils. In some embodiments, the binding affinity to an epitope within the amino acid region 125-133 of α-synuclein is 1000 times less than to the binding affinity of the antibodies to α-synuclein aggregates. In some embodiments, the antibodies do not recognize or bind to a linear epitope of α-synuclein.

In some embodiments, the antibodies disclosed herein also bind to aggregated forms of phosphorylated α-synuclein. The antibodies exhibit low binding affinity to monomeric forms of phosphorylated α-synuclein as compared to aggregated forms of phosphorylated α-synuclein, e.g. the binding affinity of the antibodies is at least 100 times less, 500 times less, 1000 times less to monomeric phosphorylated α-synuclein forms as compared to aggregated phosphorylated α-synuclein. Phosphorylation of the α-synuclein can occur at Ser129.

The antibodies disclosed herein also exhibit low binding affinity to other amyloid proteins including, but not limited to, β-synuclein, γ-synuclein monomers, IAPP (islet amyloid polypeptide), β-amyloid monomers, Tau and ABri, e.g. the binding affinity of the antibodies is at least 100 times less to one or more of these peptide/proteins than that to the α-synuclein aggregates.

In some embodiments, the binding affinity of the α-synuclein aggregate antibodies disclosed herein is at least 100 times less or at least 1000 less to β-synuclein than to α-synuclein aggregates. In particular, the binding affinity of the antibodies is at least 100 times less or at least 1000 less to β-synuclein than the binding affinity to α-synuclein fibrils.

In some embodiments, the binding affinity of the α-synuclein aggregate antibodies disclosed herein is at least 100 times less, preferably 1000 less to γ-synuclein than to α-synuclein aggregates. In particular, the binding affinities of the antibodies are at least 100 times less or at least 1000 less to γ-synuclein than to α-synuclein fibrils.

By enabling the accurate identification and quantification of disease-related α-synuclein oligomers, the diagnostic assay provided herein allows for early and precise diagnosis, as well as monitoring disease progression. These advancements allow for improving patient care and outcomes, and in guiding the development of targeted therapeutics.

The term “antibody” is herein used in the broadest sense and encompasses various antibody structures including, but not limited to, monoclonal antibodies, polyclonal antibodies, multispecific antibodies (e.g., bispecific antibodies), and antibody fragments so long as they exhibit the desired antigen-binding activity. An antibody broadly refers to any immunoglobulin (Ig) molecule comprised of heavy (H) chains and light (L) chains, or any functional fragment, mutant, variant, or derivation thereof, which retains the essential epitope-binding features of an Ig molecule. Such mutant, variant, or derivative antibody formats are known in the art, non-limiting embodiments of which are discussed below. An antibody is said to be “capable of binding” a molecule if it is capable of specifically reacting with the molecule. As used herein, the term “fragment”, when referring to an antibody should be read to mean an antigen-binding fragment. Various embodiments encompass both whole antibodies and antibody fragments, while other embodiments are limited to only whole antibodies or only antibody fragments or only one or more particular types of antibody fragment.

The term “monoclonal antibody,” as used herein, refers to an antibody obtained from identical immune cells that are clones of a unique parent cell and expressed from a particular, single encoding sequence (neglecting such variation as may arise in the expression system or cell). Typically, monoclonal antibodies are monospecific in that all of the antigen binding sites contain the same complementarity determining regions (CDRs) and thus bind to the same epitope. The modifier “monoclonal” indicates the character of the antibody as being obtained from a clonal source and is not to be construed as requiring production of the antibody by any particular method. Thus, the term encompasses antibodies obtained through traditional hybridoma technology, but also those obtained by phage display and other molecular cloning technologies.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions termed complementarity determining regions (CDRs). (See, e.g., Kindt et al. Kuby Immunology, 6th ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively.

“Antibody” encompasses “antigen-binding portion” or “antigen-binding fragment” of an antibody (or simply “antibody portion” or “antibody fragment”) and refers to a molecule other than an intact or whole antibody that comprises a portion of an intact antibody that binds the antigen to which the intact antibody binds (e.g., one or more fragments of an antibody that retain the ability to specifically bind to an antigen). Examples of antibody fragments include, but are not limited to, Fv, Fab, Fab′, Fab′-SH, F(ab′)₂, diabodies, linear antibodies, single-chain antibody molecules (e.g. scFv), heavy chain only antibodies (HCAb), and multispecific antibodies formed from antibody fragments. Papain digestion of intact antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen. However, these terms may also be applied to genetically encoded fragments of the same or similar nature, in addition to those fragments produced by proteolytic digestion. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding portion” of an antibody. Other forms of single chain antibodies, such as diabodies are also encompassed.

The “Fab” fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain that contains at least a portion of the constant region. The term includes native sequence Fc regions and variant Fc regions.

“Framework” or “FR” refers to variable domain residues other than complementarity determining region (CDR) residues. The FR of a variable domain generally consists of four FR domains: FR1, FR2, FR3, and FR4. Accordingly, the CDR and FR sequences generally appear in the following sequence in either VH or VL sequences: FR1-CD1-FR2-CDR2-FR3-CDR3-FR4.

The terms “full length antibody,” “intact antibody,” and “whole antibody” are used herein interchangeably to refer to an antibody having a structure substantially similar to a native antibody structure or having heavy chains that contain an Fc region as defined herein.

“Fv” refers to the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “class” of an antibody refers to the type of constant domain or constant region possessed by its heavy chain. There are five major classes of antibodies in humans: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. In some embodiments, recombinant technology can be used to change the isotype of an antibody.

The term “epitope” or “antigenic determinant” includes any protein or polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. Different epitopes may occupy the same or topological region of an antigen.

The present disclosure also encompasses functional equivalents of the antibodies described in this specification. Functional equivalents have binding characteristics that are comparable to those of the antibodies, and include, for example, chimerized, humanized and single chain antibodies as well as fragments thereof. Methods of producing such functional equivalents are disclosed in PCT Application WO 93/21319, European Patent Application No. 239,400; PCT Application WO 89/09622; European Patent Application 338,745; and European Patent Application EP 332,424, which are incorporated in their respective entireties by reference. Functional equivalents include polypeptides with amino acid sequences substantially the same as the amino acid sequence of the variable or hypervariable regions of the antibodies disclosed herein. “Substantially the same” as applied to an amino acid sequence is defined herein as a sequence with at least about 90%, and more preferably at least about 95% sequence identity to another amino acid sequence, as determined by the FASTA search method in accordance with Pearson and Lipman, Proc. Natl. Acad. Sci. USA 85, 2444-2448 (1988) and retaining binding specificity to the target antigen. Thus, some embodiments are functionally equivalent to, or substantially similar to, the amino acid sequence of a monoclonal antibody disclosed herein.

Some embodiments include antibodies comprising an antigen binding portion that is a functional equivalent of the antigen binding portion of the herein disclosed antibodies.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein “aggregate-specific monoclonal antibodies targeting α-synuclein (α-syn) aggregates” refers to antibodies that target early soluble “oligomers” or late insoluble “fibrils” aggregates of α-syn. In some embodiments, “aggregate-specific monoclonal antibodies targeting α-synuclein (α-syn) aggregates” also bind to the early soluble oligomers or the insoluble fibrils (i.e., the antibodies also bind to non-aggregated early soluble oligomers and late insoluble fibrils).

As used herein conformation-specific anti-α-synuclein antibodies refers to antibodies that can bind to a complex comprising aggregate-specific monoclonal antibodies targeting α-synuclein aggregates.

“Prodromal-PD” refers to the stage at which individuals do not fulfill diagnostic criteria for PD (e.g., bradykinesia and at least 1 other motor sign) but do exhibit signs and symptoms that indicate a risk of developing motor symptoms and a diagnosis of PD in the future.

“Prodromal DLB” refers to a predementia stage with signs or symptoms indicating that DLB will subsequently develop and encompasses not only cognitive deficits but also a variable mixture of noncognitive clinical features including motor symptoms and signs, sleep disorders, autonomic dysfunction, and neuropsychiatric.

“Prodromal MSA”, refers to a prodromal stage, namely a period in which some clinical signs or symptoms of disease are evident but are not yet at threshold for clinical diagnosis.

“Aggregation” or “aggregates” of α-synuclein typically involves the formation of soluble oligomers' “early aggregates”, which precede the formation of more complex “late aggregates” comprising fibrillated forms which comprise rich beta-sheet-like amyloid fibrils assemblies of the protein. The α-synuclein structure oscillates continuously between various conformations. Multiple factors such as, concentration, temperature, ionic strength, pH or mixing/agitation etc. affect the aggregation process and give rise to different species of α-syn aggregates.

“Alpha-synuclein” or “alpha-syn” or “α-syn” is a disordered dynamic protein that pathogenically aggregates into inclusion structures called Lewy bodies, Lewy neurites or glial cytoplasmic inclusions “α-synuclein pathology”. The oligomerization status of α-synuclein is typically unclear, and α-synuclein may exist predominantly as either a monomer or a variety of oligomers of different molecular weights. While the insoluble aggregates may be “final” forms of α-synuclein aggregates, recent experimental evidence (e.g., Emin, D. et al., Nat Commun. 2022 Sep. 20; 13 (1): 5512) points to smaller soluble species, usually called oligomers, as the main spreading toxic species. In some embodiments, aggregate-specific monoclonal antibodies targeting α-synuclein aggregates may also target soluble oligomers.

Some embodiments disclosed herein include a monoclonal antibody that specifically binds aggregated, non-phosphorylated α-synuclein (an anti-WT-α-syn antibody). In some embodiments, the antibody does not bind to phosphorylated α-synuclein. In some embodiments, the antibody does not bind to non-phosphorylated α-synuclein monomers. In some embodiments, the epitope recognized includes a serine residue at position 129 of α-synuclein, a site at which α-synuclein becomes phosphorylated. In some embodiments, the antibody recognizes, and can be induced by, a peptide comprising or consisting of amino acid residues 125-133 of α-synuclein (SEQ ID NO:1), namely YEMPSEEGY (SEQ ID NO: 6), for example, the peptide CYEMPSEEGY (SEQ ID NO:7). Such antibodies can be referred to as means for specifically binding to aggregated, non-phosphorylated α-synuclein. Some embodiments are compositions comprising a monoclonal antibody that binds aggregated, non-phosphorylated α-synuclein and a carrier, solvent, buffer, or other excipient. In some embodiments, the antibody is the monoclonal antibody 4B1 produced by the hybridoma 4B1, ATCC Patent Deposit Number PTA-127017.

In a facet of these embodiments, the α-synuclein aggregates are protofibrils or soluble oligomers of α-synuclein and the antibody can have high affinity for the protofibrils, the soluble oligomers, or both. In other facets of these embodiments, the α-synuclein aggregates are α-synuclein fibrils.

Disclosed herein is a method of generating an antibody that binds, or antiserum specific for, aggregated, non-phosphorylated α-synuclein comprising immunizing a mouse or other laboratory animal (for example, a rat, a hamster, or a rabbit) or an agricultural animal (for example, a goat, a sheep, or a horse) with a peptide comprising of amino acid residues 125-133 of α-synuclein (SEQ ID NO:1), namely YEMPSEEGY (SEQ ID NO: 6), for example, the peptide CYEMPSEEGY (SEQ ID NO: 7) may be referred to as means for inducing antibodies recognizing aggregated, non-phosphorylated α-synuclein. In some embodiments, the immunizing peptide is conjugated to a carrier protein, for example, keyhole limpet hemocyanin, Concholepas concholepas hemocyanin, bovine serum albumin, ovalbumin, or diphtheria or tetanus toxoid. In some embodiments, the antibody is a monoclonal antibody. In some embodiments, the method further comprises standard procedures for generating and selecting hybridomas, as are known to those of skill in the art. Some embodiments are a monoclonal antibody generated by any of these methods.

Also disclosed herein are antibodies that bind, or antiserum specific for, aggregated, non-phosphorylated α-synuclein made by a process comprising immunizing a mouse with a peptide comprising or consisting of amino acid residues 125-133 of α-synuclein (SEQ ID NO: 1), namely YEMPSEEGY (SEQ ID NO: 6), for example, the peptide CYEMPSEEGY (SEQ ID NO: 7). Some embodiments further comprise applying hybridoma technology or molecular cloning technology to obtain a monoclonal antibody. In some embodiments, the antibodies are 2A1 or 4B1.

Some embodiments are a monoclonal antibody generated by (or made by a process comprising) immunizing a laboratory animal with means for inducing antibodies recognizing aggregated, non-phosphorylated α-synuclein. In some embodiments, the laboratory animal is a mouse. In some embodiments, the means for inducing antibodies recognizing aggregated, non-phosphorylated α-synuclein are conjugated to a carrier protein. In some embodiments, the carrier protein is keyhole limpet hemocyanin. In some embodiments, the means for inducing antibodies recognizing aggregated, non-phosphorylated α-synuclein is the peptide CYEMPSEEGY (SEQ ID NO: 7). Some embodiments further comprise screening hybridomas for reactivity of the antibody against recombinant WT-α-syn. Some embodiments further comprise a counter-screens for a lack of reactivity of the antibody against one or more of pS129-α-syn, S129A-α-syn (in which serine is replaced with alanine), β-synuclein, or γ-synuclein. In some embodiments, the screening (or counter-screening) comprises an ELISA. In some embodiments, the screening (or counter-screening) comprises filter retardation assay analysis.

Some embodiments comprise a method of making a monoclonal antibody that binds aggregated, non-phosphorylated α-synuclein comprising culturing a hybridoma that secretes the antibody and collecting the culture supernatant. Some embodiments further comprise purifying the antibody from the culture supernatant. The antibodies are suitably separated from the culture medium by conventional immunoglobulin purification procedures such as, for example, proteins G-Sepharose, protein A-Sepharose, hydroxyapatite chromatography, gel electrophoresis, dialysis, or affinity chromatography. Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25 M salt). In some embodiments, the hybridoma is 4B1, ATCC Patent Deposit Number PTA-127017. Further embodiments are a monoclonal antibody that binds aggregated, non-phosphorylated α-synuclein made by any of these methods.

In the figures, discovery cohort refers to a study population (cases of disease and controls) from which samples were collected and α-synuclein aggregates was measured. Validation cohort refers to a new population recruited and having cases of disease and controls, to confirm diagnostic utility of the assays described herein.

The term “area under the curve (AUC)” refers to an index that summarizes the “overall” location of the entire ROC curve. The AUC can be interpreted as the probability that a randomly chosen diseased subject is rated or ranked as more likely to be diseased than a randomly chosen non-diseased subject. The AUC refers to the average value of sensitivity (true positive rate) against the false positive rate (1-specificity). An AUC=1 means that the diagnostic test is perfect in the differentiation between the diseased and non-diseased. An AUC=0.5 means that the curve is located on the diagonal line in ROC space and there is no predictive power. The AUCs in all the ROC curves described herein are greater than 0.5, including some that are close to 1. Thus, the assays described herein have predictive power as demonstrated herein.

Both 2A1 and 4B1 specifically target α-synuclein early “oligomers” and late “fibrils” aggregates, albeit focusing on different epitopes and forms of α-synuclein. In some embodiments, the assays described herein use the same anti-α-synuclein monoclonal antibody for both capturing and detection. Thus, the use of either a generic antibody, or an aggregate-specific antibody, enables the selective detection of α-synuclein aggregates. Described herein are results from the use of antibodies 2A1 and 4B1.

α-Synuclein seeding increases the sensitivity and accuracy of assays for detecting α-synuclein aggregates. A characteristic property of misfolded α-synuclein fibrils is the promotion of misfolding of the normal protein. In vivo, the misfolded α-synuclein fibrils spread along specific neural networks to other neurons, moving across synapses. When seeding is done in an in vitro assay, a biological sample of is co-incubated with normally folded recombinant α-synuclein, the latter misfolds, creating more aberrant protein that goes on in serial fashion to amplify the assay to the point that it may be detectable through standard histochemical techniques.

Provided herein is a diagnostic assay for detecting synucleinopathies, or α-synuclein co-pathology associated with neurodegenerative diseases, comprising: a suspended biological sample, recombinant full length or fragment of a α-synuclein monomer, or a combination thereof, in at least one well coated with an aggregate-specific monoclonal antibody targeting α-synuclein aggregates selected from 2A1 or 4B1, and measuring the amount of aggregated “seeded” α-synuclein in the biological sample.

Provided are methods for preparing diagnostic assays for detecting synucleinopathies. The assays include the steps of: a) coating wells of a plate with a first antibody targeting α-synuclein aggregates; b) adding a mixture of human biological samples and recombinant full length or fragment of a α-synuclein monomers to the coated plate to form first antibody-α-synuclein aggregate complexes; c) incubating the plate under shaking conditions or continuous mixing; d) adding labeled second anti-α-synuclein antibody to the first antibody-α-synuclein aggregate complexes and incubating; and e) detecting the first antibody-α-synuclein aggregate complexes. In some embodiments, the sample and the recombinant full-length or fragment of α-synuclein monomers are incubated together to amplify the α-synuclein aggregates prior to adding to the assay plate. In some embodiments, α-synuclein-first antibody complexes are detected with a second antibody specific for α-synuclein aggregates. The first antibody and the second antibody can be different antibodies or the same antibody.

In some embodiments, the synucleinopathies include PD, DLB, and MSA. In some embodiments the synucleinopathies is PD. In some embodiments the synucleinopathies is DLB. In some embodiments the synucleinopathies is MSA.

In some embodiments, the neurodegenerative diseases associated with α-synuclein co-pathology includes, but is not limited to, Alzheimer's disease (AD).

In some embodiments, the aggregate-specific monoclonal antibody is 2A1. In some embodiments, the aggregate-specific monoclonal antibody is 4B1.

In some embodiments, the same anti-α-synuclein monoclonal antibody (whether generic or aggregate-specific) utilized for capturing is also employed for detection, enabling the selective detection of α-synuclein oligomers.

In some embodiments, the biological samples include brain, cerebrospinal fluid, blood plasma, saliva, urine, skin, nasal swabs, or feces.

In some embodiments, the diagnostic assay further includes establishing the assay in the MSD platform using electrochemiluminescence readout.

In some embodiments, the diagnostic assay further includes adapting the assay to other immunoassay platforms, such as enzyme immunoassay, chemiluminescence assay, lateral flow immunoassay, flow cytometry, mass spectrometry, and bead-based immunoassays, including Quanterix.

Provided herein is a diagnostic assay for detecting neurodegenerative diseases characterized by protein misfolding, comprising: a suspended biological sample, recombinant full length or fragment of a α-synuclein monomer, or a combination thereof in at least one well coated with an aggregate-specific monoclonal antibody targeting α-synuclein aggregates selected from 2A1 or 4B1, and measuring the amount of aggregated α-synuclein in the biological sample.

Provided herein is a method for preparing diagnostic assays for detecting neurodegenerative diseases characterized by protein misfolding. The assays include the steps of: a) coating wells of a plate with a first antibody targeting α-synuclein aggregates; b) adding a mixture of human biological samples and recombinant full length or fragment of a α-synuclein monomer to the coated plate to form first antibody-α-synuclein aggregate complexes; c) initiating the seeding and protein amplification process incubating the plate under shaking conditions or continuous mixing; d) adding labeled second anti-α-synuclein antibodies to the first antibody-α-synuclein aggregate complexes and incubating; and e) detecting the first antibody-α-synuclein aggregate complexes. In some embodiments, the sample and the recombinant full-length or fragment of α-synuclein monomers are incubated together to amplify the α-synuclein aggregates prior to adding to the assay plate. In some embodiments, α-synuclein-first antibody complexes are detected with a second antibody specific for α-synuclein aggregates. The first antibody and the second antibody can be different antibodies or the same antibody.

In some embodiments, the neurodegenerative diseases characterized by protein misfolding include tauopathies, Alzheimer's disease, frontotemporal dementia, Pick's disease, progressive supranuclear palsy, corticobasal degeneration, Huntington's disease, amyotrophic lateral sclerosis, motor neuron diseases, spinocerebellar ataxia, and spinal muscular atrophy.

In some embodiments, the plate is coated with α-synuclein aggregate-specific monoclonal antibody 2A1. In some embodiments, the plate is coated with aggregate-specific monoclonal antibody 4B1.

In some embodiments, the biological samples include brain, CSF, blood plasma or serum, saliva, urine, skin, nasal swabs, or feces.

In some embodiments, the diagnostic assay further includes adapting the assay to other immunoassay platforms, such as enzyme immunoassay, chemiluminescence assay, lateral flow immunoassay, MSD, flow cytometry, mass spectrometry, and bead-based immunoassays, including Quanterix.

Provided herein is a method of detecting PD, DLB, or MSA in a subject comprising conducting the diagnostic assay described herein on a biological sample from the subject.

Provided herein is an immunoassay kit comprising at least one α-synuclein aggregate-specific monoclonal antibodies selected from 2A1 or 4B1. In some embodiments, the immunoassay kit includes at least one anti-α-synuclein monoclonal antibody used for capturing α-synuclein oligomers or aggregates thereof, and for detection of α-synuclein oligomers or aggregates thereof. In some embodiments, the same antibody is used for substrate capture and for complex detection. In some embodiments, different antibodies are used for substrate capture and for complex detection.

Provided herein is an antibody 2A1 having a VL sequence of SEQ ID NO:2 and a VL sequence of SEQ ID NO:3. Provided herein is an antibody 4B1 having a VL sequence of SEQ ID NO: 4 and a VL sequence of SEQ ID NO:5.

In some embodiments, the methods described herein are used to identify prodromal patients who may benefit from early administration of PD, DLB, or MSA treatments like levodopa, dopamine agonists, and/or monoamine oxidase-B inhibitors. Non-limiting examples of dopamine agonists include and are not limited to cabergoline, brocriptine, pramipexole, ropinirole, and rotigotine. Non-limiting examples of monoamine oxidase-β-inhibitors include and are not limited to selegiline, rasagiline, and safinamide. In some embodiments, such medications are taken in combination with a further therapeutic agent such as, for example, benserazide or carbidopa. In some embodiments, the antibodies described herein are used in combination with immunotherapy targeting α-synuclein aggregates, small molecules inhibiting α-synuclein aggregation, molecules inducing α-synuclein clearance, and/or other suitable strategies aimed at reducing α-synuclein expression.

In some embodiments, the described methods serve as companion tests for assessing drug engagement and monitoring the effectiveness of drugs targeting α-synuclein in clinical trials for indications such as, e.g., PD, DLB, MSA, and/or AD.

With respect to any of the antibody aspects, some embodiments are pharmaceutical compositions comprising the antibody. A pharmaceutical composition is one intended and suitable for the treatment of disease in humans. That is, it provides overall beneficial effect and does not contain amounts of ingredients or contaminants that cause toxic or other undesirable effects unrelated to the provision of the beneficial effect. A pharmaceutical composition will contain one or more active agents and may further contain solvents, buffers, diluents, carriers, and other excipients to aid the administration, solubility, absorption or bioavailability, and or stability, etc. of the active agent(s) or overall composition. A “pharmaceutically acceptable carrier, diluent, or excipient” is a medium generally accepted in the art for the delivery of biologically active agents to mammals, e.g., humans. The compounds of the present invention can be formulated as pharmaceutical compositions using a pharmaceutically acceptable carrier, diluent, or excipient and administered by a variety of routes. In particular embodiments, such compositions are for oral or intravenous administration. Such pharmaceutical compositions and processes for preparing them are well known in the art. See, e.g., REMINGTON: THE SCIENCE AND PRACTICE OF PHARMACY (A. Gennaro, et al., eds., 19.sup.th ed., Mack Publishing Co., 1995).

Aspects of the present specification provide, in part, administering a therapeutically or prophylactically effective amount of an anti-α-synuclein antibody or a pharmaceutical composition thereof. As used herein, the term “therapeutically effective amount” is synonymous with “therapeutically effective dose” and when used in reference to treating a neurodegenerative disorder associated with α-synuclein, such as Parkinson's disease, dementia with Lewy bodies, Alzheimer's disease, or multiple system atrophy, means at least the minimum dose of a compound or composition disclosed herein necessary to achieve the desired therapeutic or prophylactic effect. In some embodiments, it refers to an amount sufficient to prevent, slow, or halt the neurodegenerative process. In some embodiments, it includes a dose sufficient to reduce a symptom associated with the neurodegenerative disease. An effective dosage or amount of an anti-α-synuclein antibody or a composition thereof can readily be determined by the person of ordinary skill in the art considering all criteria (for example, the rate of excretion of the compound or composition used, the pharmacodynamics of the compound or composition used, the nature of the other compounds to be included in the composition, the particular route of administration, the particular characteristics, history and risk factors of the individual, such as, e.g., age, weight, general health and the like, the response of the individual to the treatment, or any combination thereof) and utilizing his best judgment on the individual's behalf.

The terms “treatment,” “treating”, etc., refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. Various embodiments may specifically include or exclude one or more of these modes of treatment. The herein disclosed anti-α-synuclein antibodies may be used as medicaments for the treatment of α-synuclein-associated neurodegenerative disorders. Further embodiments are methods of treating α-synuclein-associated neurodegenerative disorders comprising administering an anti-α-synuclein antibody to a subject in need thereof. In some embodiments, the α-synuclein-associated neurodegenerative disorder is Parkinson's disease, dementia with Lewy bodies, Alzheimer's disease, or multiple system atrophy. In some embodiments, the subject in need thereof is a human.

With respect to assay aspects, one embodiment is a sandwich ELISA. In such an assay, a capture antibody specific for the substance is associated with a solid support, such as a microtiter plate. A liquid containing the substance (or suspected of containing the substance, or a sample in need of determining not to include the substance) is allowed to bind to the capture antibody. Then a detection antibody, also specific for the substance, is added to allow detection of substance bound to the capture antibody. In some embodiments, the anti-WT-α-syn antibody is coated on the surface of a microtiter well or bead and used as the capture reagent and a pan-α-synuclein antibody is used as the detection reagent. In other embodiments, a pan-α-synuclein antibody is used as the capture reagent and the anti-WT-α-syn antibody is used as the detection reagent. In some embodiments, the pan-α-synuclein antibody is 11D12 (Majbour et al., Mol Neurodegener. 11, 7, 2016)

Another aspect is an immunohistochemical assay. Immunohistochemistry involves the process of selectively imaging antigens (proteins) in a tissue section by exploiting the principle of antibodies binding specifically to antigens in biological tissues. Visualizing an antibody-antigen interaction can be accomplished in a number of ways. In the most common instance, a detection antibody is used which allows the detection of the anti-α-synuclein antibody and thus the substance to which the antibody is bound.

In some embodiments, the detection antibody is a labeled antibody. In other embodiments, the anti-α-synuclein antibody is labeled. The label can include a radioactive label, an enzyme label, a colorimetric label, a fluorescent label, a chemiluminescent label, or other labels known to persons of skill in the art. In some such embodiments, the detection antibody is biotinylated so that it can bind an enzyme-linked avidin molecule, such as streptavidin conjugated with horseradish peroxidase or alkaline phosphatase. In alternative embodiments, avidin molecule is conjugated with another detectable label, for example, a fluorescent dye or quantum dot. In some embodiments, the detection antibody is directly labeled. Still further alternatives are familiar to one of skill in the art.

In further embodiments in which the anti-α-synuclein antibody is labeled, the anti-α-synuclein antibody is used for in vivo imaging by administering the labeled anti-α-synuclein antibody to a subject and detecting the label.

In some embodiments, the label is an enzymatic label such as a peroxidase (e.g., horseradish peroxidase), a galactosidase (e.g., β-D-galactosidase), or a phosphatase (e.g., alkaline phosphatase). For enzymatic labels, a substrate is needed which is cleaved by the enzyme to produce a color, fluorescence, or luminescence, which is measured spectrophotometrically. Exemplary colorimetric substrates for peroxidase include, but are not limited to, 3,3′,5,5′-tetramethylbenzidine (TMB), 3,3′,4,4′ diaminobenzidine (DAB), 4-chloro-1-naphthol (4CN), 2,2′-azino-di [3-ethylbenzthiazoline] sulfonate (ABTS), and o-phenylenediamine (OPD). In some embodiments, when the assay is an ELISA, the substrate is TMB which produces a blue color which is measured at a wavelength of 650 nm. The reaction can be halted by addition of acid or another stop reagent. Using a sulfuric acid stop solution turns TMB yellow and the color can then be read at 450 nm. Exemplary colorimetric substrates for phosphatase include, but are not limited to, 5-bromo-4-chloro-3-indolyl-phosphate/nitroblue tetrazolium (BCIP/NBT) and p-nitrophenylphosphate (p-NPP). Exemplary colorimetric substrates for galactosidase include, but are not limited to, 5-dodecanoylaminofluorescein di-β-D-galactopyranoside (C12FDG), 9H-(1,3-dichloro-9,9-dimethylacridin-2-one-7-yl), and β-D-galactopyranoside (DDAO galactoside). Exemplary fluorescent substrates include, but are not limited to, 4-methylumbelliferyl phosphate (4-MUP; for phosphatase), and 4-methylumbelliferyl galactoside (MUG; for galactosidase), fluorescein di-β-D-galactopyranoside (FDG; for galactosidase), hydroxyphenylacetic acid (HPA; for peroxidase), and 3-p-hydroxyphenylproprionic acid (HPPA; for peroxidase). Exemplary luminescent substrates include, but are not limited to, luminol, polyphenols (e.g., pyrogallol, pupurogallin, gallic acid, and umbelliferone) and acridine esters, and luciferin for peroxidase; 3-(2′-spiroadamantane)-4-methyl-4-(3′-phosphoryloxyphenyl-1, 2-dioxetane, disodium salt) (AMPPD) for phosphatase; and (3-(2′-spiroadamantane)-4-methoxy-4-(3′-β-D-galactopyranosyloxyphenyl-1,2-dioxetane (AMPGD) for galactosidase.

In some embodiments, the label is horseradish peroxidase and the substrate is TMB.

In some embodiments, the label is a colorimetric label, a fluorescent label, or a luminescent label. An exemplary colorimetric label includes, but is not limited to, nanoparticulate gold. Exemplary fluorescent labels include, but are not limited to, ethidium bromide, fluorescein and its derivatives, rhodamine and its derivatives, green fluorescent protein, Texas Red, Cascade Blue, Oregon Green, Marina Blue, an atto label, a CF™ dye, an Alexa Fluor, and a cyanine dye. Exemplary luminescent labels include, but are not limited to, luciferin and firefly luciferase.

With respect to the assay aspects, some embodiments of the assay are used as a diagnostic assay to determine whether or not an individual has a neurodegenerative disease associated with α-synuclein, for example, PD, DLB, MSA, or Alzheimer's disease. In some embodiments, the diagnostic assay comprises adding the antibody to a biological sample from a subject and detecting the presence or absence of a complex formed between α-synuclein aggregates and the antibody or fragment.

With respect to the assay aspects, some embodiments comprise a test kit comprising an antibody described herein.

### EXAMPLES

### Example 1

### Generation of Monoclonal Antibody Recognizing Non-Phosphorylated S129-α-Synuclein (WT-α-Syn) Aggregates

A short synthetic peptide, CYEMPSEEGY (SEQ ID NO:7), designed over the region of interest (amino acids 125-133 of α-synuclein; YEMPSEEGY (SEQ ID NO:6)), was used as the immunogen. This peptide was solubilized in phosphate-buffered saline (PBS) and conjugated to keyhole limpet hemocyanin (KLH) as carrier protein. Experimental procedures using mice were carried out in accordance with Laboratory Animal Research Center (LARC), Qatar University (QU), Qatar, according to the QU institutional ethical rules and regulations and approved by QU-IACUC & IBC. Specifically, female BALB/c mice (6-8 weeks old) were injected subcutaneously with the immunogen conjugate. Ten days post booster immunization, blood was collected from the tail vain and titer response was evaluated using indirect ELISA. Mice exhibiting a strong immune response were subjected to a final immunization before euthanization.

To generate hybridomas, splenocytes from immunized mice were fused with mouse myeloma cells (Sp2O-Ag14; American Type Culture Collection) at a ratio of 5:1 and fusion was induced using 50% polyethylene glycol. Fused cells were seeded in 96-well plate in IMDM media (Gibco) containing HAT (Sigma). Positive clones were transferred to 24-well plates and screened multiple times to ensure stability.

For the indirect ELISA, a 96-well clear plate was coated with recombinant WT-α-syn and incubated overnight at 4° C. The following day, the plate was blocked with blocking buffer (PBST containing 2.25% gelatin) for 1 hour at RT. The plate was then washed three times with PBST, and antisera from the mice, or culture supernatants, were added in serial dilutions. Next, the plate was washed and goat anti-mouse IgG-HRP (1:20 K, Jackson ImmunoResearch) was added. The plate was washed again and detected with TMB substrate (Abcam). Following color development, the reaction was stopped by addition of 0.6 N H₂SO₄ and the absorbance was measured at 450 nm.

For isotyping, culture supernatant was screened against anti-mouse heavy chain antibodies (Isotyping Kit, Sigma-Aldrich) in ELISA and IgG-positive clones were selected. The IgG clones were then subjected to single-cell cloning. Wells with single clones were grown to confluency and screened at least three times for further selection of stable clones. Selected clones were grown in CDM4mAb media (Hyclone) to confluency. Culture supernatant was then collected and purified using protein-G agarose affinity chromatography (Sigma-Aldrich). Several monoclonal antibodies were produced, purified and thoroughly characterized. Two antibodies, 2A1 and 4B1, were selected for further characterization.

The purity of the 4B1 was assessed using SDS-PAGE under reducing conditions. The specificity for recombinant WT-, pS129-, or mutated S129A-α-syn (with a substitution of serine 129 to alanine) proteins was assessed by Western blot. The data showed that 4B1 is specific for WT-α-syn and does not recognize pS129-α-syn. Furthermore, there was no evident band when serine was replaced with alanine (S129A-α-syn) indicating that S129 is an integral residue of the 4B1's target epitope. Western blot analysis showed that 4B1 recognized α-synuclein sparing β- and γ-syn. The antibodies recognized both human and mouse α-synuclein to an equal extent. Additionally, 2A1 and 4B1 reacted equally to WT-α-syn from human or mouse species.

The purity of the antibody 2A1 was assessed using SDS-PAGE under reducing conditions. The specificity of 2A1 for the aggregated α-synuclein was assessed by filter retardation assay. The data showed that 2A1 is specific for α-synuclein aggregates (oligomers or fibrils) and did not recognize α-synuclein monomers. Additionally, 2A1 reacted equally to α-synuclein aggregates from human or mouse species.

Hybridoma 4B1 was deposited with the American Type Culture Collection, 10801 University Boulevard, Manassas VA, 20110-2209 on Jun. 24, 2021 and given the Patent Deposit Number (accession number) PTA-127017.

The amino acid sequences of the 2A1 and 4B1 antibodies are provided in Table 1.

TABLE 1

| SEQ ID NO: | Sequence Name | Amino Acid Sequence |
|---|---|---|
| 2 | mAb 2A1 VL | MRVLAELLGLLLFCFLGVRCDIQMNQSPSSLSASLGDTI |
| | | TITCHASQNINVWLSWYQQKPGNIPKLLIYKASNLHTGV |
| | | PSRFSGSRSGTGFTLTISSLQPEDIATYYCQQGQSFPWT |
| | | FGGGTKLEIKRADAAPTVSIFPPSSEQLTSGGASVVCFL |
| | | NNFYPKDINVKWKIDGSERQNGVLNSWTDQDSKDSTYSM |
| | | SSTLTLTKDEYERHNSYTCEATHKTSTSPIVKSFNRNEC |
| 3 | mAb 2A1 VH | MGWSCIILFLVATATGVHSQVQLQQPGAELVKPGASVKM |
| | | SCKASGYTFSSYNLHWVKQTPGQGLEWIGAIYPGNGDTS |
| | | YNQKFKGKATLTADKSSSTAYMQLSSLTSEDSAVYYCTR |
| | | RDYYGSRGFAYWGQGTLVTVSAAKTTPPSVYPLAPGSAA |
| | | QTNSMVTLGCLVKGYFPEPVTVTWNSGSLSSGVHTFPAV |
| | | LQSDLYTLSSSVTVPSSTWPSETVTCNVAHPASSTKVDK |
| | | KIVPRDCGCKPCICTVPEVSSVFIFPPKPKDVLTITLTP |
| | | KVTCVVVDISKDDPEVQFSWFVDDVEVHTAQTQPREEQF |
| | | NSTFRSVSELPIMHQDWLNGKEFKCRVNSAAFPAPIEKT |
| | | ISKTKGRPKAPQVYTIPPPKEQMAKDKVSLTCMITDFFP |
| | | EDITVEWQWNGQPAENYKNTQPIMDTDGSYFVYSKLNVQ |
| | | KSNWEAGNTFTCSVLHEGLHNHHTEKSLSHSPGK |
| 4 | mAb 4B1 VL | MKLPVRLLVLMFWIPASSSDVVMTQTPLSLPVSLGDQAS |
| | | ISCRSSQSLVHSNGNTYLHWYLQKPGQSPKLLISKVSNR |
| | | FSGVPDRFSGSGSGTDFTLKISRVEAEDLGVYFCSQSTH |
| | | VPLTFGAGTKLELKRADAAPTVSIFPPSSEQLTSGGASV |
| | | VCFLNNFYPKDINVKWKIDGSERQNGVLNSWTDQDSKDS |
| | | TYSMSSTLTLTKDEYERHNSYTCEATHKTSTSPIVKSFN |
| | | RNEC |
| 5 | mAb 2A1 VH | MNFGLSLIFLVPVLKGVLCEVKLVESGGGLVQPGGSLKL |
| | | SCAASGITFSSYTMSWVRQTPEKRLEWVGYISNGGGRTY |
| | | YPDTVKGRFTISRDNAKNTLYLQMSSLKSEDTAMYYCAR |
| | | QITTAMGYYYAMDYWGQGTSVTVSSAKTTPPSVYPLAPG |
| | | SAAQTNSMVTLGCLVKGYFPEPVTVTWNSGSLSSGVHTF |
| | | PAVLQSDLYTLSSSVTVPSSTWPSETVTCNVAHPASSTK |
| | | VDKKIVPRDCGCKPCICTVPEVSSVFIFPPKPKDVLTIT |
| | | LTPKVTCVVVDISKDDPEVQFSWFVDDVEVHTAQTQPRE |
| | | EQFNSTFRSVSELPIMHQDWLNGKEFKCRVNSAAFPAPI |
| | | EKTISKTKGRPKAPQVYTIPPPKEQMAKDKVSLTCMITD |
| | | FFPEDITVEWQWNGQPAENYKNTQPIMDTDGSYFVYSKL |
| | | NVQKSNWEAGNTFTCSVLHEGLHNHHTEKSLSHSPGK |

Using filter retardation assay, an assay that preserves the protein conformation, surprisingly we found that 4B1 only recognized WT-α-syn aggregates, and did not recognize the monomeric form of α-syn protein. Syn-02 (a mouse monoclonal antibody against α-syn aggregates (Vaikath et al., Neurobiol Dis. 79:81-99, 2015)), and Syn-1 were used as control antibodies.

### Example 2

### Assays

### Assay Methodology

Human biofluids (BH/CSF/blood) along with recombinant full-length, N-terminally truncated, C-terminally truncated, or N- and C-terminally truncated monomeric α-synuclein, were suspended in the sample buffer and added to wells of the plates coated with aggregate-specific anti-α-synuclein mAbs (e.g., 2A1 or 4B1). The plate was incubated under shaking conditions, and following a washing step, biotinylated conformation-specific anti-α-synuclein antibodies (e.g., 2A1 or 4B1) were added to corresponding wells and incubated for 1 hour. Next, the plate after washing was incubated for an hour with streptavidin-HRP followed by a final wash. TMB substrate solution was added, and the plate is incubated in the dark for 10 mins after which stop solution was added to stop the reaction. The plate is read at 450 absorbance using microplate reader.

The assay was established in the MSD platform which uses electrochemiluminescence readout. Another example setup that may be adapted is the use of magnetic beads coated with the aggregate-specific monoclonal antibody instead of multi-well plates. Samples would be incubated with these beads along with the recombinant α-synuclein monomers, then incubated with intermittent shaking initiating the seeding and protein amplification process. Following a washing step, biotinylated detection antibody (e.g., anti-α-synuclein antibody) may be added to corresponding wells and incubated for 1 hour. The plates may then be washed and incubated with streptavidin-phycoerythrin (SA-PE) for 1 hour, washed again and re-suspended in assay buffer, then the plate is read using Bio Plex-3D system (Bio-Rad).

The assay, using the antibodies disclosed herein are applicable to any immunoassay setup/platforms such as enzyme immunoassay, chemiluminescence assay, lateral flow immunoassay, MSD, flow cytometry, mass spectrometry, bead-based immunoassays such as Quanterix etc.

In sum, a quantitative high through-put immunoassay for synucleinopathies with a high diagnostic potential for use in diagnostics, longitudinal studies and clinical trials has been developed. The assay provides at least the following advantages:
i) highly specific;
ii) compatible with different biological samples (Brain lysates, CSF, blood, saliva, urine, skin homogenates, nasal swabs or feces);
iii) low volume of sample required;
iv) fast turnaround time;
v) relatively simple test that could be conducted in any clinical/research lab.

### Results

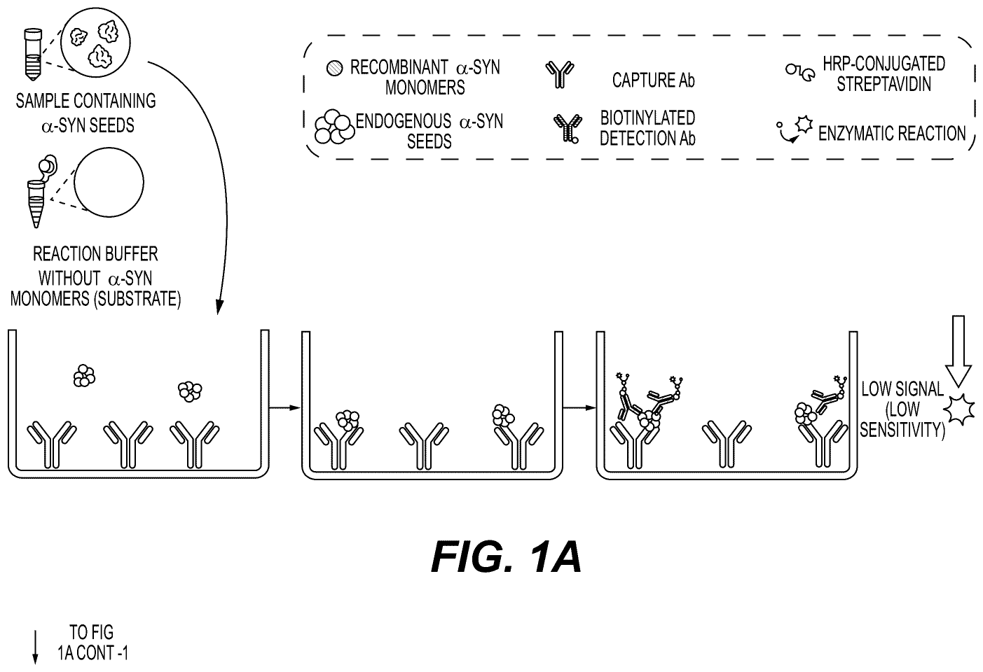

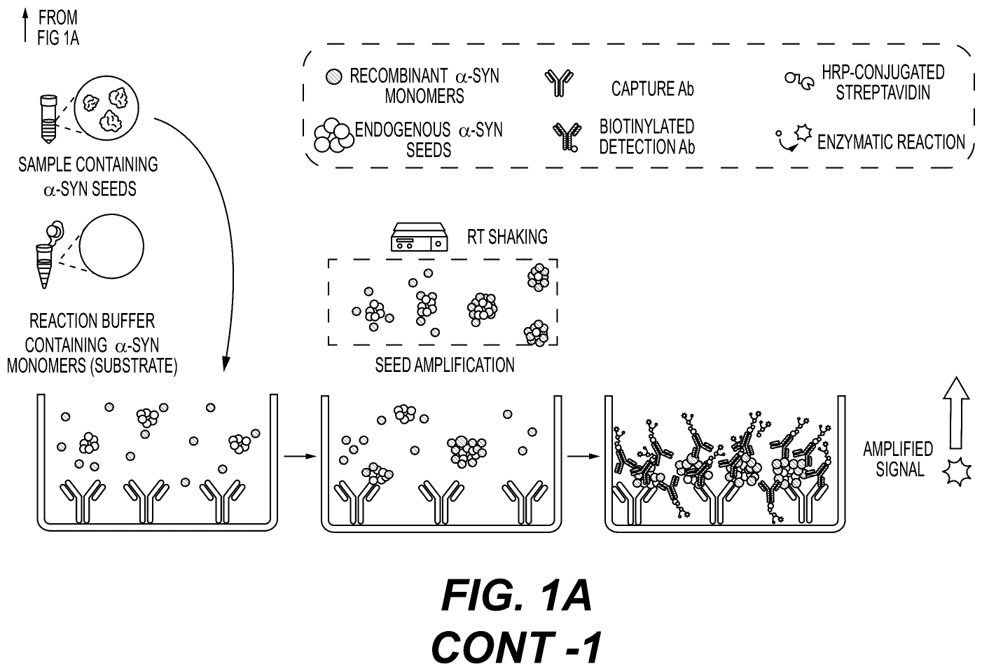

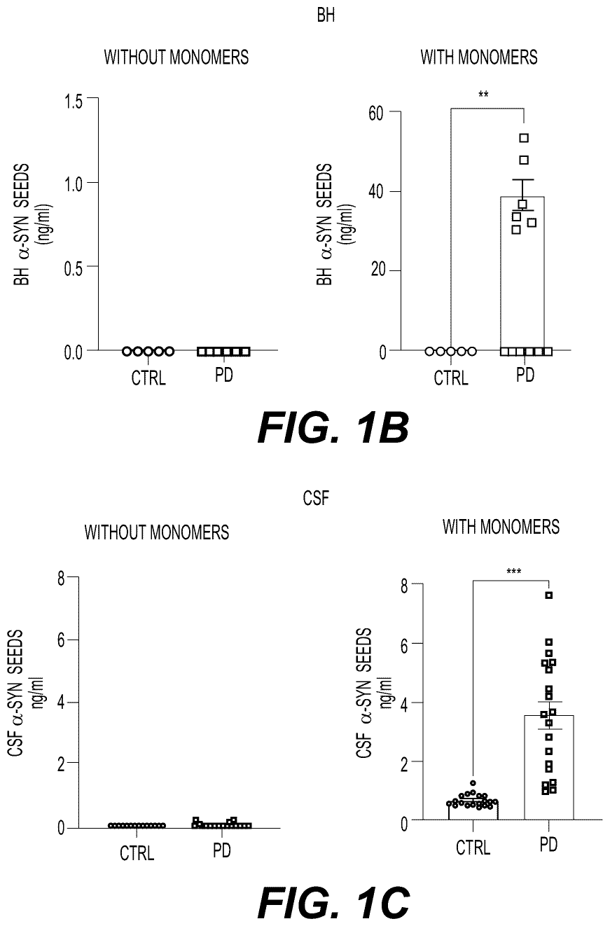

Experimental results are described in FIGS. 1-5. With respect to ROC curve analysis, a ROC curve lying on the diagonal line reflects the performance of a diagnostic test that is no better than chance level, i.e. a test which yields the positive or negative results unrelated to the true disease status. A ROC curve corresponding to progressively greater discriminant capacity of diagnostic tests is located progressively closer to the upper lefthand corner in “ROC space.”

FIG. 1A shows a diagrammatic representation version of the ELISA assay. The top panel of the diagram represents a conventional sandwich ELISA protocol that employs a conformation-specific anti-α-synuclein mAb (2A1) without the seeding amplification step. In this setup, samples are processed in a reaction buffer without adding recombinant α-synuclein monomer to serve as a substrate. Although this method allows for the capture and detection of endogenous α-synuclein seeds within the samples, it achieves this with limited sensitivity.

The bottom panel illustrates an enhancement to this method under identical experimental conditions (same reaction buffer, shaking, and temperature). By introducing α-synuclein monomers, there is an amplification of endogenous α-synuclein seeds. This amplification can occur through two possible mechanisms: either directly in solution, leading to the formation of a complex aggregate of recombinant and endogenous alpha-synuclein captured by the monoclonal antibody, or by the initial capture of endogenous seeds by the antibody followed by seeding. The amplification significantly increases the number of potential binding sites available for the detection antibody, biotinylated 2A1, thereby substantially enhancing the sensitivity and the overall signal detected in the assay.

FIG. 1B shows a scatter plot that illustrates the seeding effect of α-synuclein seeds observed in a pilot study of BH samples from PD patients and healthy controls.

FIG. 1C shows a scatter plot that illustrates the seeding effect of α-synuclein seeds observed in a pilot study of CSF samples from PD patients and healthy controls.

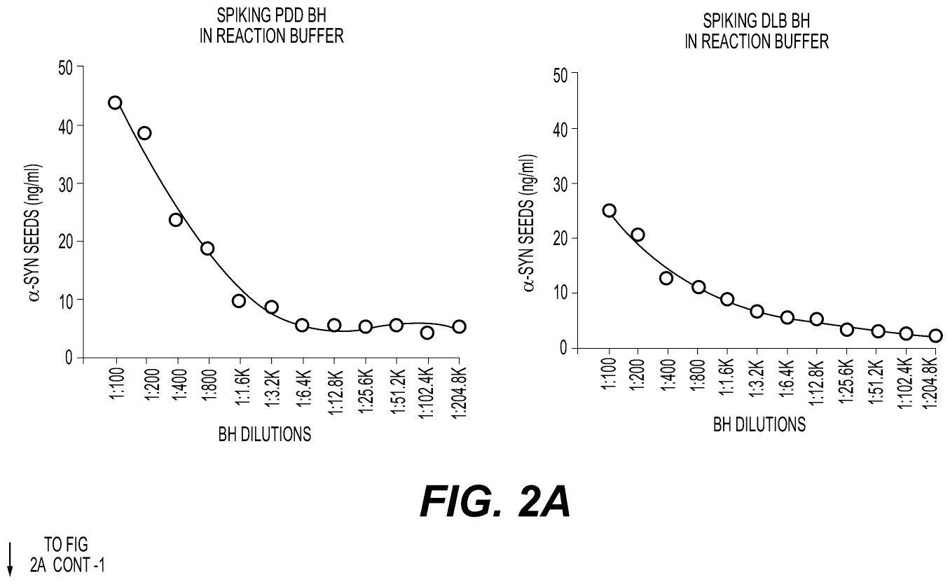

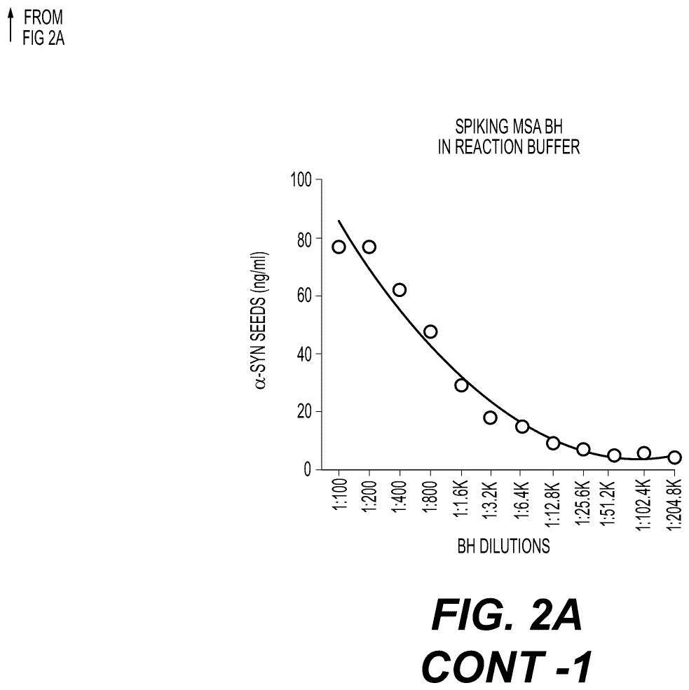

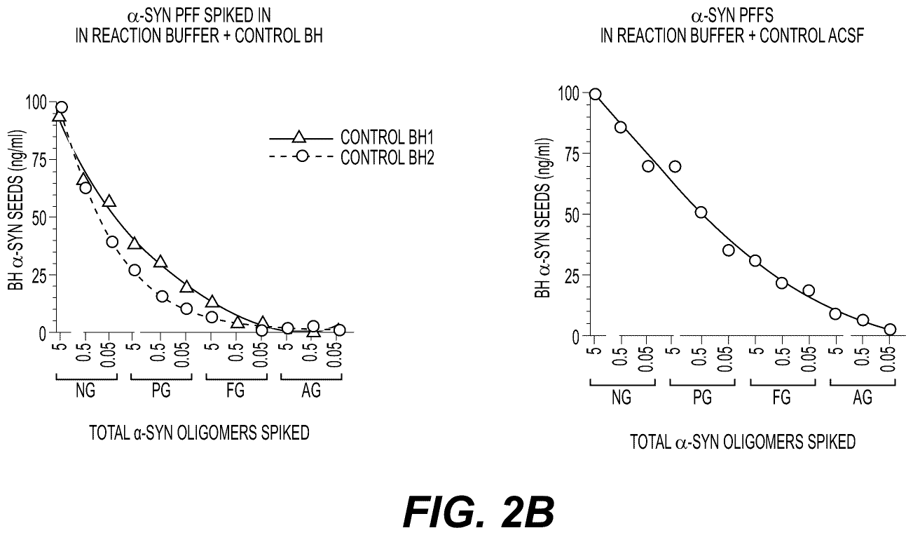

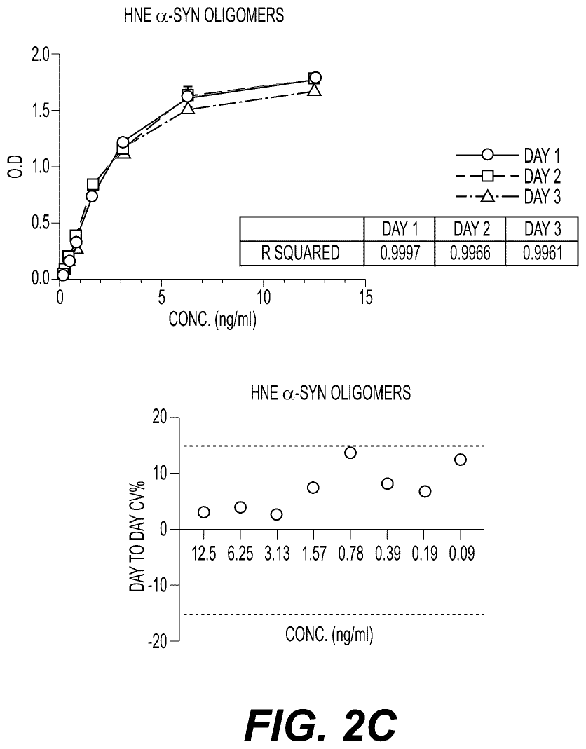

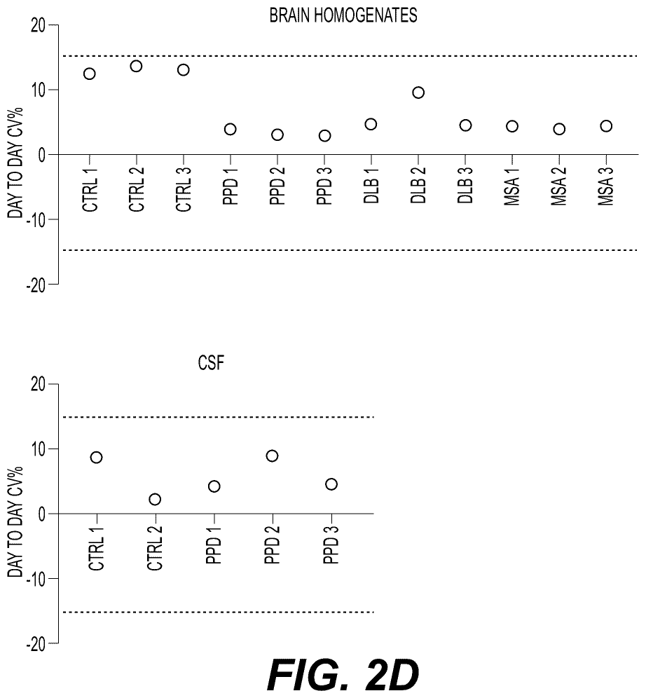

FIG. 2A illustrates the sensitivity of the assays described herein by using different dilutions, between 1:200 and 1:2042000, of BH obtained from PD, DLB and MSA subjects, wherein seeded α-synuclein can be effectively detected. Sensitivity was established up to approximate dilution of 1:64000 of BH samples.

FIG. 2B shows testing of α-synuclein PFFs at different concentrations in control BH and artificial CSF (ACSF). The assays described herein demonstrated sensitivity levels at 0.5 femtogram in BH and 0.5 attogram in ACSF buffer.

FIG. 2C shows inter-assay variability plots of HNE α-synuclein oligomer standards tested over three non-consecutive days. The inter-assay CV % is no more than about 15% and the optical density is about the same over three non-consecutive days.

FIG. 2D shows inter-assay variability plots of BH and CSF samples tested over two non-consecutive days. The inter-assay CV % is no more than about 15% in BH samples and no more than about 10% in CSF samples.

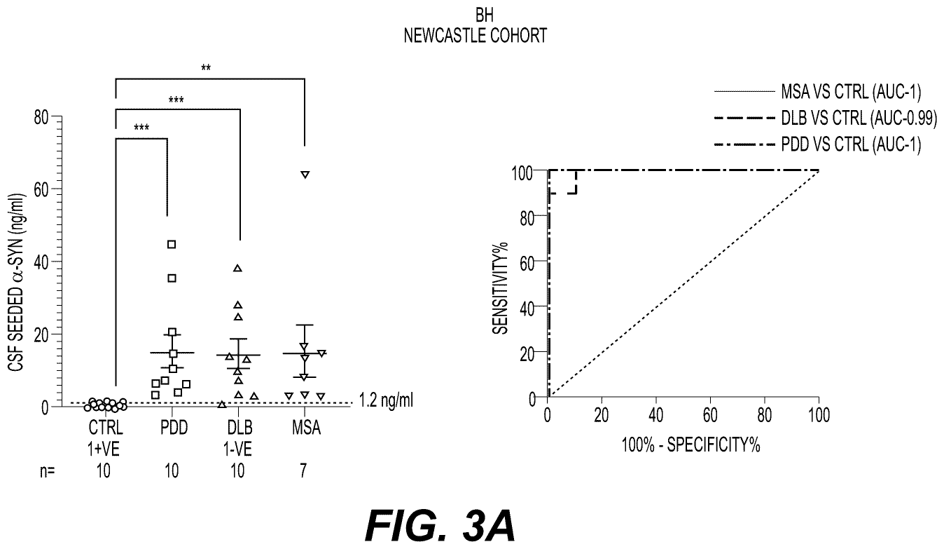

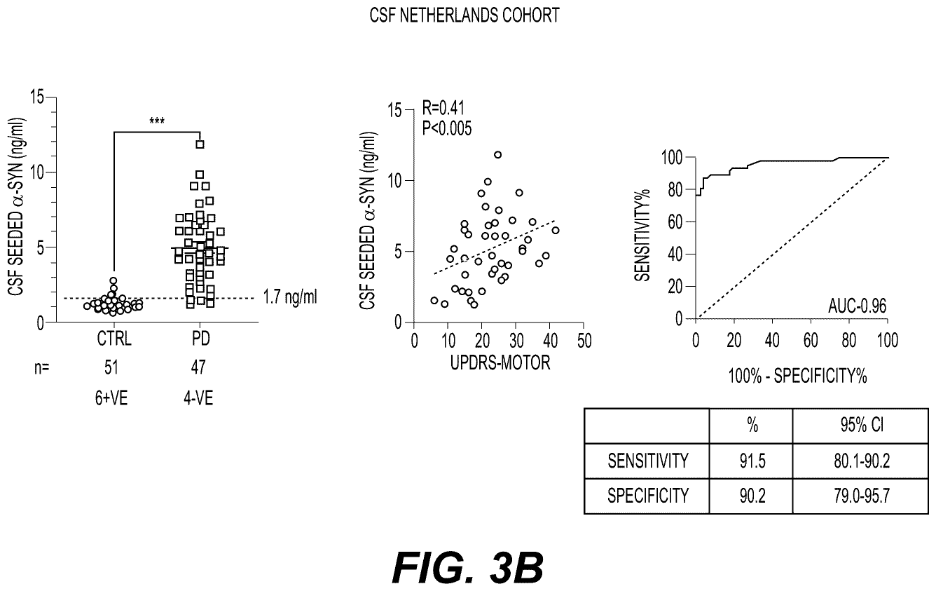

FIG. 3A shows assay validation in a larger cohort of samples of BH from different synucleinopathies PDD, DLB, and MSA. Scatter plot and ROC analysis of measured seeded α-synuclein levels in BH samples of PD, DLB, MSA vs control. The ROC curves are close to the upper left hand corner confirming the diagnostic discriminant capacity of the assays described herein.

FIG. 3B shows a scatter plot depicting the measured levels of seeded α-synuclein in CSF samples from a larger cohort consisting of PD patients and healthy controls and a scatter plot showing measured levels of seeded α-synuclein in CSF samples correlated to PD motor score UPDRS III. The ROC curves are close to the upper left hand corner confirming the diagnostic discriminant capacity of the assays described herein.

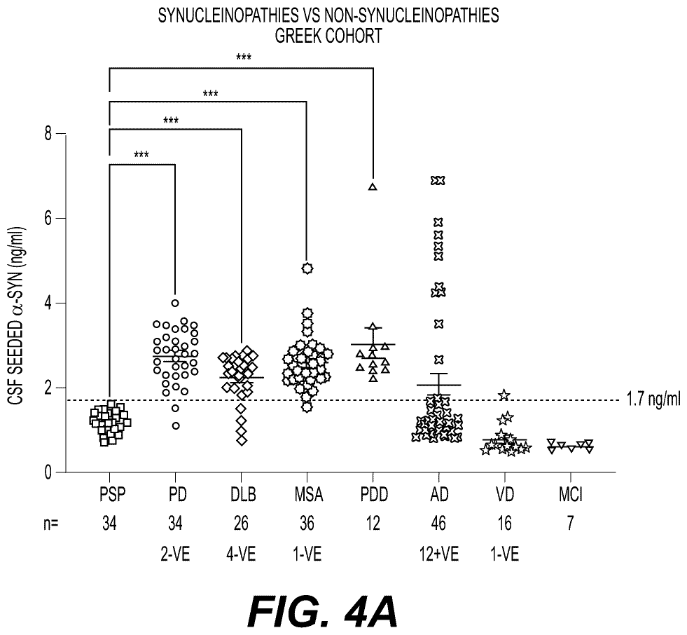

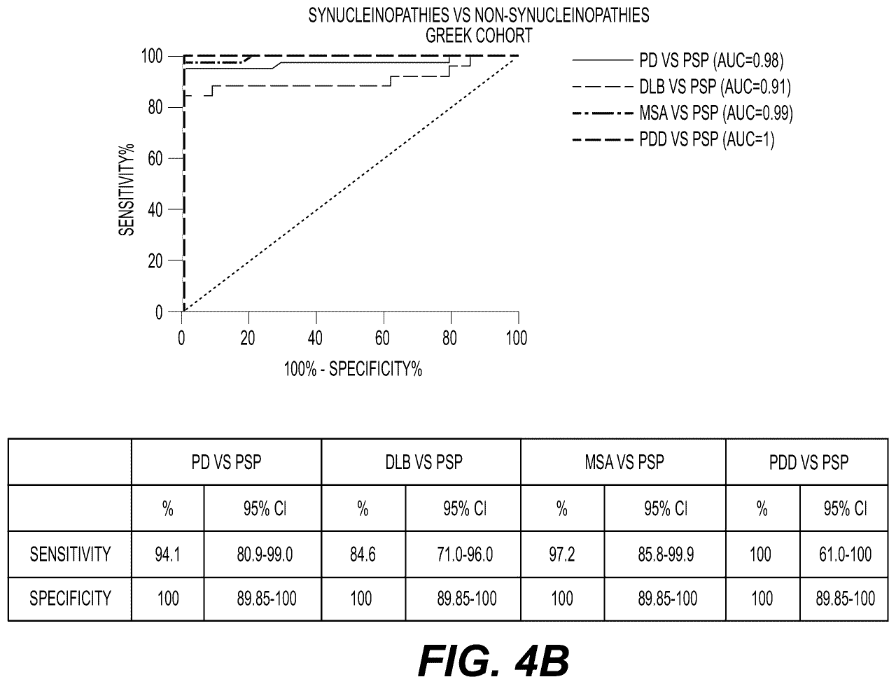

FIG. 4A shows immunoassay specificity for synucleinopathies with a scatter plot of measured seeded α-synuclein levels in CSF samples from synucleinopathies (PD, MSA, DLB, PDD) compared to non-synucleinopathies (progressive supranuclear palsy (PSP), Alzheimer's Disease (AD), vascular dementia (VD), and mild cognitive impairment (MCI)).

FIG. 4B shows receiver operating characteristic (ROC) curve analysis of diagnostic performance of assay in PD, PDD, DLB, MSA vs PSP. The ROC curves are close to the upper left hand corner confirming the diagnostic discriminant capacity of the assays described herein.

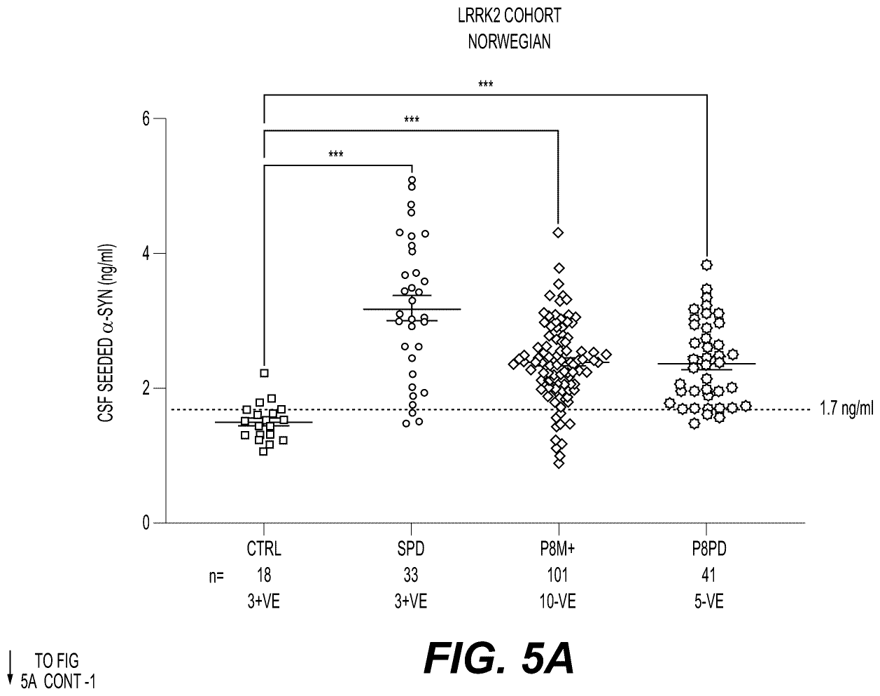

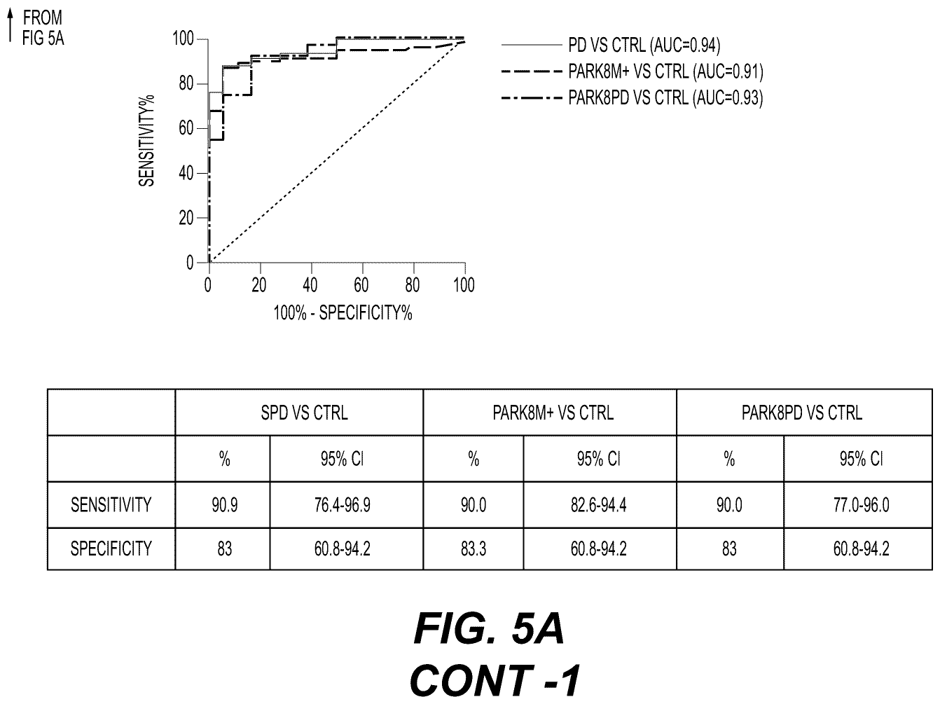

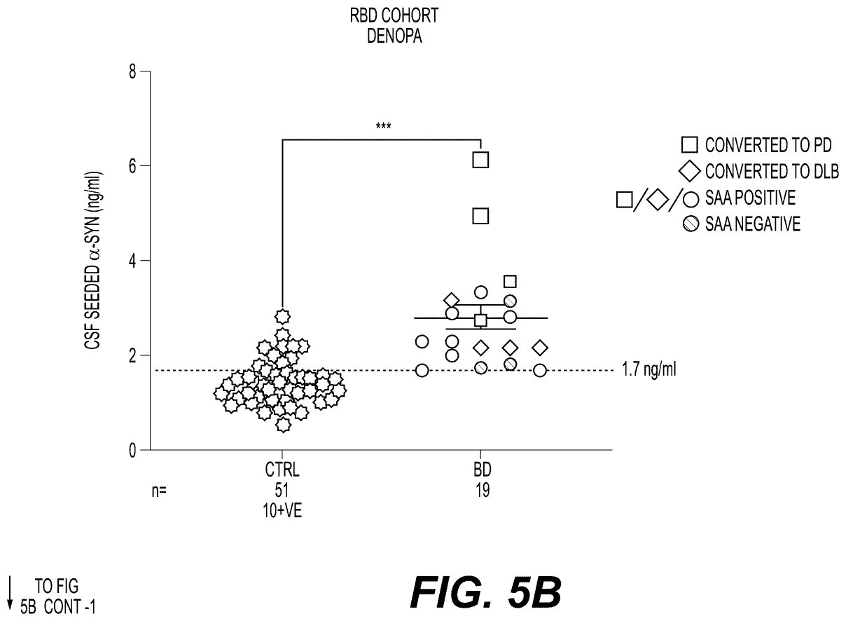

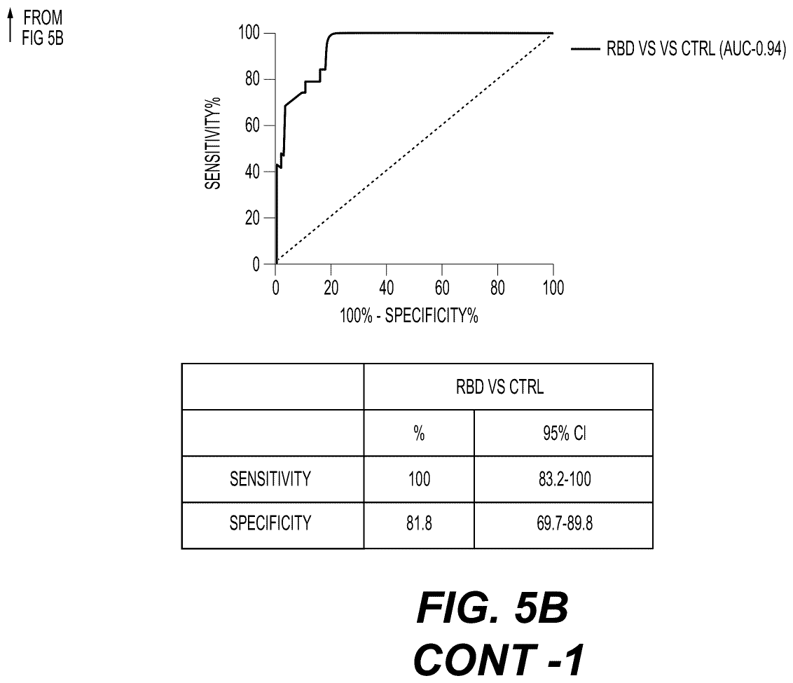

FIG. 5A shows scatter plot and ROC curve analyses of seeded α-synuclein in validation cohort of CSF samples from LRRK2 cohort with sPD, manifesting (P8PD) non-manifesting (P8M+) LRRK2 mutation carriers vs healthy controls. The ROC curves are close to the upper left hand corner confirming the diagnostic discriminant capacity of the assays described herein.

FIG. 5B shows scatter plot and ROC curve analyses of seeded α-synuclein in patients with RBD. The ROC curves are close to the upper left hand corner confirming the diagnostic discriminant capacity of the assays described herein in detecting prodromal α-synucleinopathies.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

Furthermore, references to patents and printed publications may have been made in this specification. Each of the above-cited references and printed publications are individually incorporated herein by reference in their entirety.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

What is claimed is:

1. An antibody, or fragments thereof, specific for α-synuclein aggregates, wherein the antibody has a variable light (VL) and a variable heavy (VH) amino acid sequence comprising:
(a) a VL sequence of SEQ ID NO:2 and a VH sequence of SEQ ID NO:3; or
(b) a VL sequence of SEQ ID NO:4 and a VH sequence of SEQ ID NO:5.

2. The antibody, or fragments thereof, wherein the antibody comprises a VL sequence of SEQ ID NO:2 and a VH sequence of SEQ ID NO:3.

3. A method for detecting α-synuclein aggregates in a sample from a subject, the method comprising:
a) coating wells of a plate with a first antibody targeting α-synuclein aggregates;
b) adding a mixture of a human biological sample and recombinant full-length or fragment of α-synuclein monomers to the coated plate to form first antibody-α-synuclein aggregate complexes;
c) initiating the seeding and protein amplification process by incubating the plate under shaking conditions or continuous mixing; and
d) detecting the antibody-α-synuclein aggregate complexes;
wherein the first antibody targeting α-synuclein aggregates is an antibody according to claim 1.

4. The method of claim 3, wherein the α-synuclein-first antibody complexes are detected with a second antibody specific for α-synuclein aggregates.

5. The method of claim 4, wherein the first antibody and the second antibody are different antibodies or the same antibody.

6. The method of claim 3, wherein the synucleinopathies is selected from Parkinson's disease, dementia with Lewy bodies, and multiple system atrophy.

7. The method of claim 3, wherein at least one of the first or second antibody comprises a VL sequence of SEQ ID NO:2 and a VH sequence of SEQ ID NO:3.

8. The method of claim 3, wherein at least one of the first or second antibody comprises a VL sequence of SEQ ID NO:4 and a VH sequence of SEQ ID NO:5.

9. The method of claim 3, wherein the sample is selected from brain, cerebrospinal fluid, blood plasma or serum, saliva, urine, skin, nasal swab, or feces.

10. A method for detecting a neurodegenerative disease characterized by protein misfolding in a subject, the assay comprising:
a) coating wells of a plate with a first antibody targeting α-synuclein aggregates;
b) adding a mixture of human biological samples and recombinant full length or fragment of α-synuclein monomers to the coated plate to form first antibody-α-synuclein aggregate complexes;
c) initiating the seeding and protein amplification process by incubating the plate under shaking conditions or continuous mixing; and
d) detecting the first antibody-α-synuclein aggregate complexes;
wherein the first antibody targeting α-synuclein aggregates is an antibody according to claim 1.

11. The method of claim 10, wherein the α-synuclein-first antibody complexes are detected with a second antibody specific for α-synuclein aggregates.

12. The method of claim 11, wherein the first antibody and the second antibody are different antibodies or the same antibody.

13. The method of claim 10, wherein the neurodegenerative diseases characterized by protein misfolding is selected from tauopathies, Alzheimer's disease, frontotemporal dementia, Pick's disease, progressive supranuclear palsy, corticobasal degeneration, Huntington's disease, amyotrophic lateral sclerosis, motor neuron diseases, spinocerebellar ataxia, psychosis, schizophrenia, Creutzfeldt-Jacob disease, and spinal muscular atrophy.

14. The method of claim 10, wherein the sample is selected from brain, cerebrospinal fluid, blood plasma or serum, saliva, urine, skin, nasal swab, and feces.

15. The method of claim 10, wherein the antibody comprises a VL sequence of SEQ ID NO:2 and a VH sequence of SEQ ID NO:3.

16. A method for treating a neurodegenerative disorder with α-synuclein pathology in a subject in need thereof comprising administering an antibody, or fragment thereof, according to claim 1 to the subject.

17. The method of claim 16, wherein the neurodegenerative disorder with α-synuclein pathology is selected from Parkinson's disease, Alzheimer's disease, dementia with Lewy bodies, and multiple system atrophy.

18. The method of claim 16, wherein the antibody comprises a VL sequence of SEQ ID NO:2 and a VH sequence of SEQ ID NO:3.


SEQUENCE LISTING (7 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 234 aa, synthetic construct
MRVLAELLGLLLFCFLGVRCDIQMNQSPSSLSASLGDTITITCHASQNINVWLSWYQQKPGNIPKLLIYKASNLHTGVPSRFSGSRSGTGFTLTISSLQPEDIATYYCQQGQSFPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 463 aa, synthetic construct
MGWSCIILFLVATATGVHSQVQLQQPGAELVKPGASVKMSCKASGYTFSSYNLHWVKQTPGQGLEWIGAIYPGNGDTSYNQKFKGKATLTADKSSSTAYMQLSSLTSEDSAVYYCTRRDYYGSRGFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 4 - protein, 238 aa, synthetic construct
MKLPVRLLVLMFWIPASSSDVVMTQTPLSLPVSLGDQASISCRSSQSLVHSNGNTYLHWYLQKPGQSPKLLISKVSNRFSGVPDRFSGSGSGTDFTLKISRVEAEDLGVYFCSQSTHVPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - protein, 466 aa, Synthetic construct
MNFGLSLIFLVPVLKGVLCEVKLVESGGGLVQPGGSLKLSCAASGITFSSYTMSWVRQTPEKRLEWVGYISNGGGRTYYPDTVKGRFTISRDNAKNTLYLQMSSLKSEDTAMYYCARQITTAMGYYYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 6 - protein, 9 aa, Homo sapiens
YEMPSEEGY

SEQ ID NO: 7 - protein, 10 aa, synthetic construct
CYEMPSEEGY